(19) United States
(12) Patent Application Publication
(10) Pub. No.: US 2023/0002340 A1
(43) Pub. Date: Jan. 5, 2023

(54) SEPARATION OF CANNABINOIDS FROM MIXTURES THEREOF BY DISTILLATION

(71) Applicant: CANOPY GROWTH CORPORATION, Smiths Falls (CA)

(72) Inventors: Christopher ADAIR, Smiths Falls (CA); Ben GEILING, Smiths Falls (CA); Logan MARYNISSEN, Smiths Falls (CA)

(21) Appl. No.: 17/631,591

(22) PCT Filed: Jul. 30, 2020
(86) PCT No.: PCT/CA2020/051051

§ 371 Date: Jan. 31, 2022

Related U.S. Application Data
(60) Provisional application No. 62/881,038, filed on Jul. 31, 2019.

(51) Int. Cl. C07D 311/80; C07C 37/74; B01D 3/14; B01D 3/00; B01D 1/22; B01D 5/00

(52) U.S. Cl. CPC C07D 311/80; C07C 37/74; B01D 3/148; B01D 3/008; B01D 1/222; B01D 5/006

(57) ABSTRACT

The present disclosure relates to isolating one or more cannabinoids from an input mixture. There is disclosed an apparatus that comprises a volatizing unit, a fractional distillation unit, and a condensing unit. The volatizing unit receives and volatilizes the input mixture to provide a cannabinoid-containing vapor stream and a residue. The fractional distillation unit comprises a plenum for receiving the cannabinoid-containing vapor stream and separates a first cannabinoid from at least a second cannabinoid. The condensing unit is configured to receive a portion of the cannabinoid-containing vapor stream comprising the first cannabinoid from the plenum and to form a condensed first cannabinoid output stream and a recirculate stream. There are also disclosed methods for isolating one or more cannabinoids employing a recirculate stream.

### CROSS-REFERENCE TO RELATED APPLICATION

This application claims priority to and benefit of U.S. Provisional Patent Application Ser. No. 62/881,038 filed on Jul. 31, 2019, which is hereby incorporated by reference.

### TECHNICAL FIELD

The present disclosure generally relates to the isolation of cannabinoids and mixtures of cannabinoids. In particular, the present disclosure relates to separation of cannabinoids from cannabinoid-containing resins or concentrates to isolate pure compounds or purified mixtures.

### BACKGROUND

Naturally available cannabinoids, such as phytocannabinoids that are derived from plants, are typically sourced as a mixture of various cannabinoid compounds. Other plant components include, but are not limited to, lipids such as triglycerides and waxes. Due to the complexity of the mixture, it is challenging to fully understand the pharmacologic effect of individual cannabinoids and mixtures thereof. Furthermore, isolating individual cannabinoids from a source mixture has also proven challenging. Known methods for separating cannabinoids from each other in a mixture of cannabinoids include chromatography, short path distillation, and crystallization.

In chromatography, an input material is dissolved in a solvent to form a mobile phase. The mobile phase passes through a chromatography column. When the mobile phase containing the dissolved mixture passes through the chromatography column, the compounds within the mobile phase can travel at different speeds. The amount of time it takes for a compound to travel through the column is called retention time and it is a result of size, shape, total charge, hydrophobic groups present on the compound's surface, and binding capacity with the chromatography column. A widely used chromatography method for purification of molecules is High Pressure Liquid Chromatography (HPLC). This method involves passing the mobile phase through a chromatography column under pressures ranging from 10-400 atm and at a high flow rate. During HPLC, the use of small particles and the application of a high pressure increases the separation of the compounds within the mobile phase, which results in a shorter analysis time. However, HPLC requires complex and expensive equipment, generates significant waste, and is costly because of the large quantities of organic solvent required. Generally speaking, organic solvents present specific and further challenges because they can be expensive, toxic if consumed, and they are typically highly flammable and, therefore, they require specific equipment to handle safely. Other forms of chromatography, such as flash chromatography, simulated moving bed chromatography, and centrifugal partition chromatography can all suffer from similar challenges as HPLC with respect to complexity and solvent requirements.

Short path distillation involves a distillate travelling a short distance and it is typically performed under high vacuum to allow for the separation of larger heat-sensitive compounds. This method can run continuously and has the advantage of not using solvents to achieve the desired separation. However, short path distillation relies on a significant difference in vapor pressure between the compounds being separated. While this vapor pressure difference may hold true between cannabinoid compounds, as a whole, versus other components of plant-derived mixtures, the separation of individual cannabinoids from each other, or even from some waxes and oils, is not readily achievable by short path distillation.

Crystallization is another methodology that is known for separating cannabinoids from a mixture. Crystallization is possible with some cannabinoid compounds in the neutral form (primarily cannabigerol and cannabidiol) and the carboxylic acid form of all cannabinoids. Crystallization is challenging because it often takes considerable time. Crystallization also uses organic solvent and it is typically only able to produce isolates/single compounds—not complex mixtures. Furthermore, crystallization is traditionally a batch style process, which limits its utility or even prevents its integration into a continuous processing method. Crystallization also requires a high purity input prior to beginning the process in order to maximize yields. This means that an alternate separation technology is typically used before crystallization, which results in an overall inefficiency in the separation methodology.

Each of the known methodologies for separating cannabinoids from a mixture are challenged by inefficient and incomplete separation of desired compounds from the mixture, high operational costs and, often times, the necessity of using organic solvents. However, given the potential pharmacological value of individual cannabinoids, improved separation methods to provide highly pure individual cannabinoids—or mixtures of cannabinoids with specific relative ratios of one compound to another compound—may be desirable.

### SUMMARY

The present disclosure relates to apparatus and methods for isolating one or more cannabinoids from an input mixture.

In an embodiment, the present disclosure relates to an apparatus for isolating one or more cannabinoids from an input mixture, the apparatus comprising: (a) a volatizing unit that is configured to receive and volatilize the input mixture into a cannabinoid-containing vapor stream and a residue; (b) a fractional distillation unit that is in fluid communication with the volatizing unit, the fractional distillation unit comprising a column that defines a plenum for receiving the cannabinoid-containing vapor stream, wherein the plenum is configured to separate a first cannabinoid within the cannabinoid-containing vapor stream from at least a second cannabinoid; and (c) a condenser unit that is configured to receive a portion of the cannabinoid-containing vapor stream comprising the first cannabinoid from the plenum and to form a condensed first cannabinoid output stream and a recirculate stream.

In an embodiment, the apparatus disclosed herein further comprises a distributor that is configured to receive the recirculate stream and to conduct the recirculate stream to the plenum.

In an embodiment, the present disclosure relates to a method for isolating one or more cannabinoids from an input mixture, the method comprising steps of: (a) volatilizing the input mixture to provide a cannabinoid-containing vapor stream and a residue; (b) conducting the cannabinoid-containing vapor stream to a fractional distillation unit; (c) separating a first cannabinoid from at least a second cannabinoid within the fractional distillation unit; (d)

conducting a portion of the cannabinoid-containing vapor stream comprising the first cannabinoid to a condenser unit; (e) condensing the conducted portion of the cannabinoid-containing vapor stream within the condenser unit to provide a condensed first cannabinoid product stream and a recirculate stream; (f) optionally repeating steps (a) to (e) using the recirculate stream; and (g) collecting a product from the fractional distillation unit, wherein the product comprises at least the second cannabinoid.

In an embodiment, the input mixture is a cannabis concentrate. In an embodiment, the input mixture is a cannabis resin.

Without being bound by any particular theory, carboxylic acid-containing cannabinoids can undergo heat-induced chemical transformations that can include decarboxylation. Free carbon dioxide gas formation can occur during a decarboxylation reaction. When heat-induced decarboxylation occurs during separation by fractional distillation, the change in pressure caused by the liberated gas can result in a change in effective boiling points of the cannabinoids. Complete separation of cannabinoids by fractional distillation can be unsuccessful under changing pressures. Performing more than one iteration of fractional distillation may result in improved separation and produce substantially pure yields of one or more cannabinoids from the mixture. Furthermore, condensing a portion of the more volatile compounds present within the fractional distillation column may provide a further valuable product stream and the option to recirculate a stream from the condensing process back into the fractional distillation column for further processing and isolation of desirable products.

### BRIEF DESCRIPTION OF DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a schematic representation of an apparatus, according to an embodiment of the present disclosure, which comprises a volatizing unit in fluid communication with a fractional distillation unit.

FIG. 2 shows a schematic representation of an embodiment of the fractional distillation unit shown in FIG. 1 wherein the unit includes a condenser unit and devices to control temperature and pressure and a plenum that houses traditional column packing materials.

FIG. 3 shows a schematic representation of another apparatus, according to an embodiment of the present disclosure that includes a preliminary treatment unit that is upstream of the volatizing unit.

FIG. 4 shows a schematic representation of a preliminary treatment, according to at least one embodiment of the present disclosure, for use with the apparatus shown in FIG. 3.

FIG. 5 shows a schematic representation of a method according to the present disclosure.

FIG. 6 shows a line graph that represents the Residue Product (RP) ratio of CBD:THC plotted over the various distillation temperatures for Example 4.

FIG. 7 shows a line graph that represents the net weight versus the temperature of the residue and the distillate for Example 5.

FIG. 8 shows data for Example 6, wherein FIG. 8A shows a line graph that represents the enrichment of CBD:THC over the four trials; and, FIG. 8B shows a line graph that represents the percentage of CBD (upper line) and THC (lower line) in the distillate product.

FIG. 9 shows a histogram that represents the distillate net output compared with the pressure within the distillation unit for Example 7.

FIG. 10 shows a histogram that represents the distillate/residue split as compared to the feed rate for Example 7.

FIG. 11 shows a line graph that represents the distillate/residue split as compared to the various temperatures tested for Example 8.

### DETAILED DESCRIPTION

Embodiments of the present disclosure relate to at least one apparatus and at least one method for separating one or more cannabinoids from an input mixture.

Some embodiments of the present disclosure relate to an apparatus that comprises a volatizing unit that is in direct fluid communication with a fractional distillation unit. Some embodiments of the present disclosure relate to a method that comprises the steps of volatilizing the input mixture and separating one cannabinoid from one or more other cannabinoids within the input mixture. The embodiments of the present disclosure may be suitable for use on an industrial scale and may have the advantages of being continuously operated while substantially reducing or avoiding the use of organic solvents.

In one aspect, the present disclosure relates to an apparatus for isolating one or more cannabinoids from an input mixture.

In an embodiment, the apparatus comprises: (a) a volatizing unit that is configured to receive and volatilize an input mixture into a cannabinoid-containing vapor stream and a residue; (b) a fractional distillation unit that is in fluid communication with the volatizing unit, the fractional distillation unit comprising a column that defines a plenum for receiving the cannabinoid-containing vapor stream, wherein the plenum is configured to separate a first cannabinoid within the cannabinoid-containing vapor stream from at least a second cannabinoid; and (c) a condenser unit that is configured to receive a portion of the cannabinoid-containing vapor stream comprising the first cannabinoid from the plenum and to form a condensed first cannabinoid output stream and a recirculate stream.

As used herein, the term “volatizing unit” refers to a unit or component that can volatilize or evaporate a substance. The volatizing unit can be of any suitable structure for volatilizing the input mixture. In an embodiment, the volatizing unit may be an evaporator, a wiped film evaporator, a short path distillation unit, a rising-falling film evaporator, a pot still, a jacketed or heated vessel, a centrifugal evaporator, a centrifugal short path distillation, or any combination thereof. In some embodiments of the present disclosure, the volatizing unit is a wiped film evaporator, such as an LCI LabVap® Thin-Film Evaporator that includes a heated surface and a rotating wiping system that physically agitates the input mixture therein for heat and mass transfer of the one or more cannabinoids to a vapor state. In some embodiments, the volatizing unit is a VKL70-5 Shortpath Distillation System for causing a heat and mass transfer of the one or more cannabinoids to a vapor state.

As used herein, the term “fractional distillation unit” refers to a unit or component that physically separates a mixture into one or more component parts or fractions based on boiling point of the component parts. In an embodiment, the fractional distillation unit may comprise a distillation column. The distillation column may be of any suitable shape or size including, but not limited to, a vertical cylindrical column or a distillation tower. In an embodiment, the fractional distillation unit comprises a temperature control unit and/or a pressure control unit. In this regard, in certain embodiments, the distillation unit can be configured to perform both evaporation and condensation.

In an embodiment, the fractional distillation column defines a plenum that may house packing material, or not. In an embodiment, the plenum is configured to separate one or more cannabinoids from at least one other cannabinoid. In an embodiment, the plenum is configured to separate a first cannabinoid from at least a second cannabinoid, at least a second and third cannabinoid, at least a second, third or fourth cannabinoid, or at least an even greater number of cannabinoids. In an embodiment, the plenum is configured to separate multiple cannabinoids from other cannabinoids. In an embodiment, plenum is configured to separate 1, 2, 3, 4, 5, 6, 7, 8, 9, 10 or more cannabinoids.

As used herein, the term “condenser unit” refers to a unit or component that condenses a gaseous or vapor state into a liquid state through cooling. The condenser unit may be of any suitable shape, size and material, and may for example be of industrial scale or lab scale. In an embodiment, the condenser is air-cooled, water-cooled, evaporative, or a combination thereof. In some embodiments, the condenser may be connected at the column head of the fractional distillation unit to condense a vaporized fraction (e.g. a lighter vaporized fraction).

As used herein, the term “recirculate stream” refers to a portion of the condensate provided by the condenser unit that is conducted to the plenum, the volatizing unit, or both. In an embodiment, a distributor is configured to receive the recirculate stream and to conduct the recirculate stream back to the plenum. The distributor may be of any suitable shape, size and material. In an embodiment, a reflux splitter modulates the distributor.

As used herein, the term “cannabinoid” refers to: (i) a chemical compound belonging to a class of secondary compounds commonly found in plants of genus cannabis; and/or (ii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC),Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), M-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-05), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

As used herein, the term “THC” refers to tetrahydrocannabinol. “THC” is used interchangeably herein with “Δ9-THC”.

Structural formulae of cannabinoids of the present disclosure may include the following:

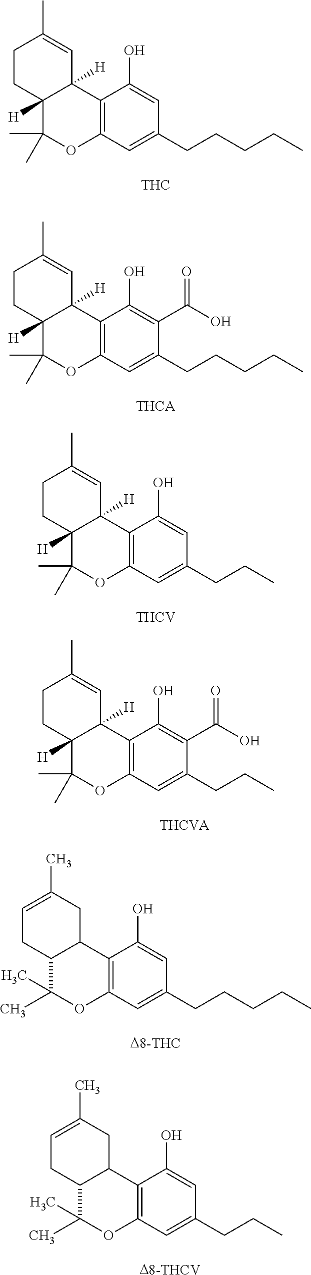

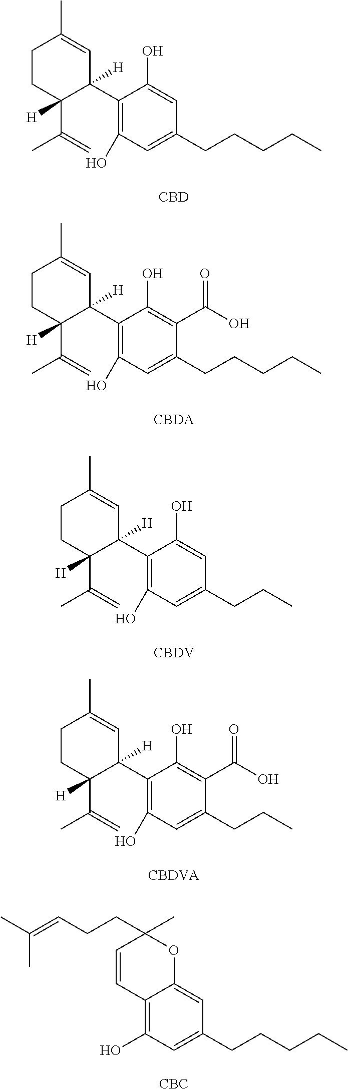

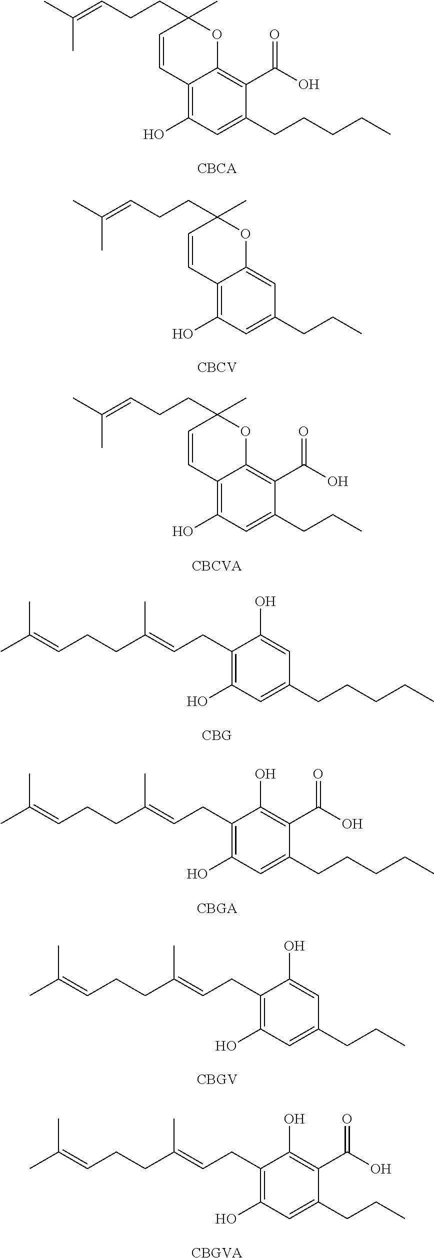

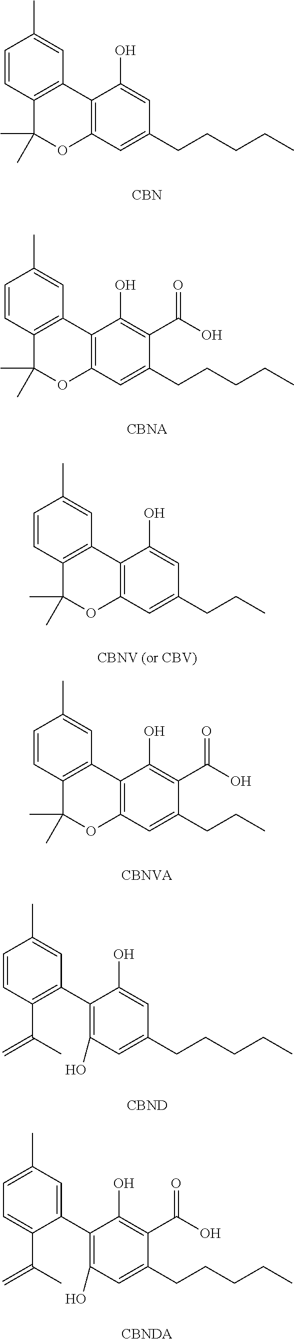

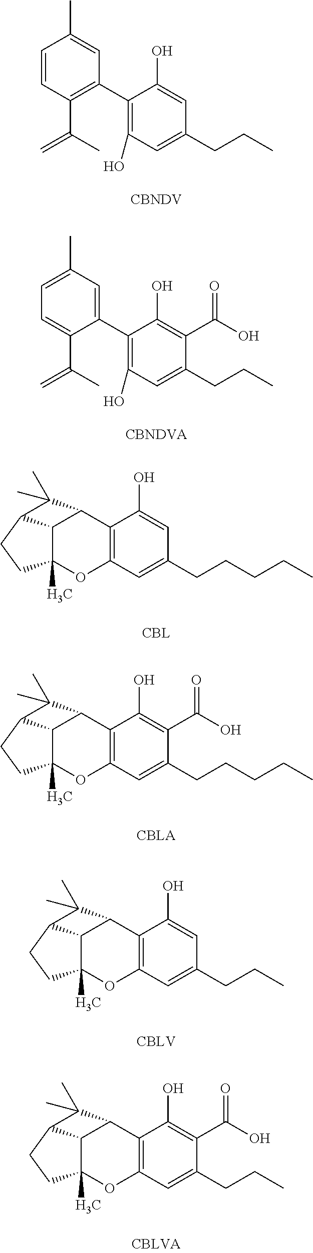

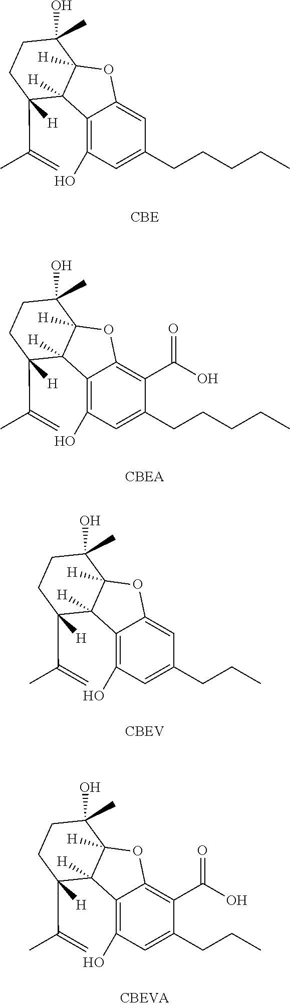

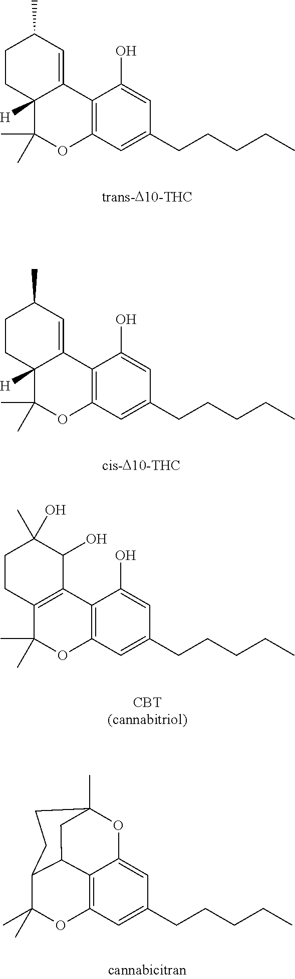

In the context of the present disclosure, the terms “first cannabinoid”, “second cannabinoid”, “third cannabinoid”, “fourth cannabinoid”, and so on, include and encompass any cannabinoid, including any of those described herein.

In select embodiments, the cannabinoid isolated by the apparatus and methods disclosed herein (e.g. the first cannabinoid and the at least second cannabinoid) may for example and without limitation be any of those described herein. In a particular, embodiment, the cannabinoid isolated by the apparatus and methods disclosed herein may be THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

In select embodiments of the present disclosure, the first cannabinoid and the at least second cannabinoid may comprise CBD, CBDV, CBC, CBCV, CBG, CBGV, THC, THCV, or a regioisomer thereof. As used herein, the term “regioisomers” refers to compounds that differ only in the location of a particular functional group.

In an embodiment, the first or the at least second cannabinoid is THC.

In an embodiment, the first or the at least second cannabinoid is CBD.

As used herein, the term “input mixture” includes any cannabis plant material or extract thereof. Where the apparatuses or methods herein describe a “first input mixture” and a “second input mixture”, it is the first input mixture that is the original source material (i.e. plant material or extract thereof), whereas the subsequent input mixtures (e.g. second or third) are a mixture formed in accordance with the described methods using a first input mixture. In an embodiment, the input mixture is a cannabis concentrate.

As used herein, “cannabis concentrate” refers to a mixture of cannabinoids that is obtained from a cannabis plant, such as for example a mixture of compounds or compositions that have been extracted from cannabis. Non-limiting embodiments of a cannabis concentrate include a cannabis distillate, a cannabis isolate, a cannabis resin, or any other type of extract containing one or more cannabinoids or terpenes, or both. In an embodiment, the input mixture is a cannabis resin.

In some embodiments, the apparatus as disclosed herein may be used to separate or a first, second, third, fourth, or more cannabinoids from any number of other cannabinoids. In this manner, a purified mixture of cannabinoids may be prepared.

In another aspect, the present disclosure relates to methods of isolating one or more cannabinoids from an input mixture (e.g. a cannabis resin).

In an embodiment, the present disclosure relates to a method for isolating one or more cannabinoids from an input mixture, the method comprising steps of: (a) volatilizing the input mixture to provide a cannabinoid-containing vapor stream and a residue; (b) conducting the cannabinoid-containing vapor stream to a fractional distillation unit; (c) separating a first cannabinoid from at least a second cannabinoid within the fractional distillation unit; (d) conducting a portion of the cannabinoid-containing vapor stream comprising the first cannabinoid to a condenser unit; (e) condensing the conducted portion of the cannabinoid-containing vapor stream within the condenser unit to provide a condensed first cannabinoid product stream and a recirculate stream; (f) optionally repeating steps (a) to (e) using the recirculate stream; and (g) collecting a product from the fractional distillation unit, wherein the product comprises at least the second cannabinoid.

The volatilizing may be done by any suitable means to cause at least one cannabinoid in the input mixture to vaporize. In an embodiment, the volatilizing can be by an evaporator, a wiped film evaporator, a short path distillation unit, a rising-falling film evaporator, a pot still, a jacketed or heated vessel, a centrifugal evaporator, a centrifugal short path distillation or combinations thereof. In a particular embodiment, the volatilizing is by a wiped film evaporator as described elsewhere herein.

The conducting may be by any suitable means including, but not limited to, passing the input mixture through a conduit. Non-limiting examples of conduits include tubing and piping. The conduit may be of any suitable size and material.

The condensing may be by any suitable means to provide a condensed first cannabinoid product stream and a recirculate stream, such as by the condenser unit described elsewhere herein.

In the methods disclosed herein, a recirculate stream is provided by the condenser unit. In some embodiments, the recirculate stream may be conducted back to the volatizing unit, the distillation unit, or both. Having been previously volatilized the recirculate stream comprises cannabinoids in the decarboxylated form, and recirculating this product back into the distillation unit provides improved methods for isolating cannabinoids.

In an embodiment of the methods herein, the recirculate stream may be fed back to the volatizing unit to undergo a further volatilization. In some embodiments, the recirculate stream may be combined with an additional quantity of input mixture, or not. In some embodiments, the recirculates stream may be fed back into the distillation unit. Here, typically being comprised of the lighter fraction and comprising compounds with lower boiling points, volatilization of the recirculate stream often occurs.

Optionally then, steps (a) to (e) of the methods disclosed herein may be repeated any number of times including use of the recirculate stream. In some embodiments, the recirculate stream is used in the volatilizing step, with or without fresh input mixture. In other embodiments, the recirculate stream may be fed directly to the distillation unit for further separation of cannabinoids. Advantageously, the separation is more controllable due to the substantial absence of acidic cannabinoids. In the methods disclosed herein, a distributor may be used to direct the flow of the recirculate stream, including by use of a reflux splitter.

The collecting may be by any suitable means to obtain all or a portion of product containing the cannabinoid. In an embodiment, the collecting of product that comprises a cannabinoid may be by a distillation tray. A distillation tray may facilitate condensation of one or more cannabinoids. In an embodiment, the collecting is by a suitable container, such as a round-bottom flask or collection flask.

In the context of the present disclosure, the quantity of a cannabinoid in a particular composition may be expressed in various fashions, such as but not limited to a percentage of the total weight of the particular composition or as a ratio that represents the relative quantity of a cannabinoid as compared to another compound within the particular composition. Additionally, the relative quantities of two cannabinoids (for example a first cannabinoid relative to a second cannabinoid) in a particular composition may be expressed as a ratio (first cannabinoid:second cannabinoid). The relative quantities of cannabinoid products in a mixture may be referred to with analogous ratios (e.g. second cannabinoid:third cannabinoid). Those skilled in the art will recognize that a variety of analytical methods may be used to determine such quantities and ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof.

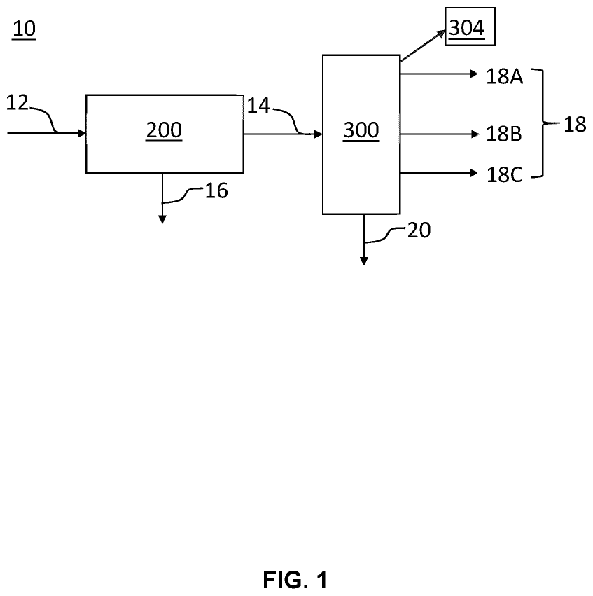

Embodiments of the present disclosure will now be described in detail including references to FIG. 1 to FIG. 5, which show embodiments of the apparatus and methods according to the present disclosure.

FIG. 1 shows a non-limiting example of an embodiment of an apparatus 10 that comprises a volatizing unit 200, a fractional distillation unit 300 (which may also be referred to as a rectifier unit), and a condenser unit 304. In this embodiment, the volatizing unit 200 may be any number of apparatus that can receive an input mixture 12. The input mixture 12 may be comprised of one or more cannabinoids as described herein and other non-cannabinoid components.

The volatizing unit 200 may be any number of apparatus that can receive the input mixture 12. In an embodiment, the volatizing unit 200 may be configured to volatilize a portion of the input mixture 12 to produce a cannabinoid-containing vapor stream 14 and a residue 16. In the context of the present disclosure, the term “residue” refers to the input mixture remaining in the volatizing unit after volatilizing to provide the cannabinoid-containing vapor stream.

For example, the volatizing unit 200 may apply heat, and optionally physical agitation, to the input mixture 12 for producing the cannabinoid-containing vapor stream 14 and the residue 16. It will be appreciated by those skilled in the art that the cannabinoid-containing vapor stream 14 may comprise cannabinoids and, optionally, further constituents of the input mixture 12. In some embodiments, substantially most, substantially all, or all of the cannabinoids within the input mixture 12 are volatilized and entrained in the cannabinoid-containing vapor stream 14. The residue 16 may be substantially free of cannabinoids, or not. In some embodiments, the residue 16 may be conducted back (not shown) to join the input mixture 12.

In some embodiments, the volatizing unit 200 may be a wiped film evaporator, a short path distillation unit, a rising-falling film evaporator, a pot still, a jacketed or heated vessel, a centrifugal evaporator, a centrifugal short path distillation or combinations thereof. In some embodiments of the present disclosure, the volatizing unit 200 is a wiped film evaporator, such as an LCI LabVap® Thin-Film Evaporator, that includes a heated surface and a rotating wiping system that physically agitates the input mixture 12 therein for heat and mass transfer of the one or more cannabinoids to a vapor state.

In some embodiments, the cannabinoid-containing vapor stream 14 can be conducted, for example through one or more fluid conduits, to the distillation unit 300 for separating one or more cannabinoids from other constituents of the cannabinoid-containing vapor stream 14 (e.g. other cannabinoids). The distillation unit 300 can be configured to separate heat sensitive compounds within the input mixture where such compounds have a relatively narrow boiling-point difference (FIG. 1). In some embodiments of the present disclosure, the distillation unit 300 can be a fractional distillation unit that is configured to separate at least the first cannabinoid from the remainder of the other cannabinoids and other constituents within the cannabinoid-containing vapor stream 14. In some embodiments of the present disclosure, the fractional distillation unit 300 can be configured to separate more than one cannabinoid from the other constituents within the cannabinoid-containing vapor stream 14 for producing at least one cannabinoid output stream 18, which may also be referred to as a distillate stream or a product, and a waste stream 20. In some embodiments, the waste stream 20 may be conducted back (not shown) to join the input mixture 12. While FIG. 1 shows three cannabinoid output streams 18A, 18B and 18C, the skilled person will appreciate that there may be more or less cannabinoid output streams produced by the fractional distillation unit 300. The skilled person will also appreciate that the at least one cannabinoid output stream 18 may also comprise one or more output streams of a cannabinoid.

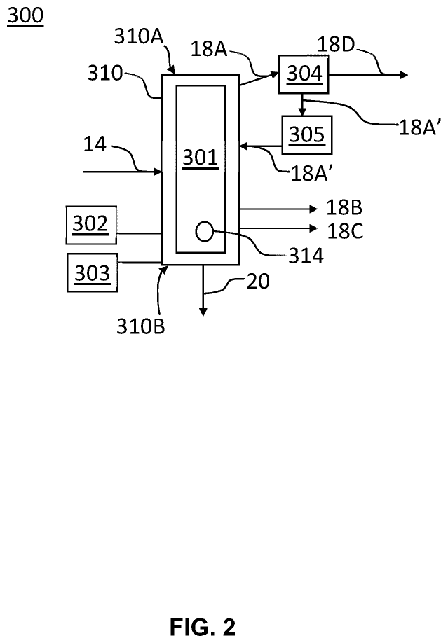

As shown in FIG. 2, the fractional distillation unit 300 may comprise a column 310 with a first end 310A, which may also be referred to as the column head, and a second end 310B. Together these two ends 310A, 310B define an internal plenum 301. The plenum 301 houses one or more separation trays (not shown) that are configured to separate at least one cannabinoid from within the cannabinoid-containing vapor stream 14 from other constituents of the cannabinoid-containing vapour stream 14, which may include at least a second cannabinoid, a third cannabinoid, a fourth cannabinoid and further cannabinoids.

In some embodiments, the fractional distillation unit 300 may comprise both a temperature control unit 302 and pressure control unit 303. The temperature control unit 302 can be configured to heat or cool the temperature within the plenum 301. In some embodiments of the present disclosure, the column 310 may be sealed at each end so that the plenum is isolated from the ambient pressure and the pressure control unit 303 is configured to increase or decrease the pressure within the plenum 301. In some embodiments of the present disclosure, the continuous fractional distillation unit 300 operates at a temperature of between about 100° C. and about 275° C. or between about 120° C. and about 150° C. within a range of pressures of between about 0.001 mbar and 110 mbar.

In some embodiments, the plenum 301 may house a packing material 314. The packing material 314 may include, but is not limited to, at least one of the following: MONTZ-Pak Type A3-750, MONTZ-Pak Type A3-1000, MONTZ-Pak Type A3-1200, MONTZ-Pak Type A3-1500, MONTZ-Pak Type A3-1900, Sulzer laboratory packing DX, or any combination thereof.

In an embodiment, fractional distillation unit 300 comprises a condenser 304 that is operatively connected at, near, or to the first end 310A (see FIG. 2). The condenser 304 can be configured to condense a lighter vaporized cannabinoid fraction (for example the first cannabinoid output stream 18A, which may for example be the most volatile cannabinoid within the mixture cannabinoids) to form a condensed first cannabinoid product 18D. Optionally, a portion of the condensed distillate 18A′ can be conducted from the condenser 304 to one or more distributors 305. The distributors 305 may be configured to recirculate at least part of the portion of the condensed stream 18A′ back into the plenum 301 (recirculate stream) where the portion of the condensed stream 18A′ can mix with the cannabinoid-containing stream 14 within the distillation unit 300. The second cannabinoid-containing output stream 18B and potentially the third cannabinoid-containing output stream 18C (and potentially others) can also be collected from the distillation unit 300.

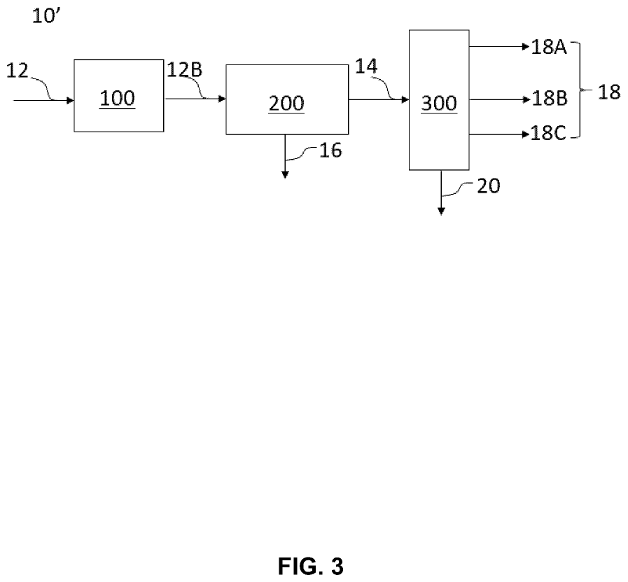

As shown in the non-limiting example of FIG. 3, some embodiments of the present disclosure relate to an apparatus 10′ that is configured to process the input mixture 12 for producing a processed second input mixture 12B. The apparatus 10′ has many of the same components as the apparatus 10, with at least one difference being the processing unit 100. In these embodiments, the processed second input mixture 12B may be conducted to the volatizing unit 200.

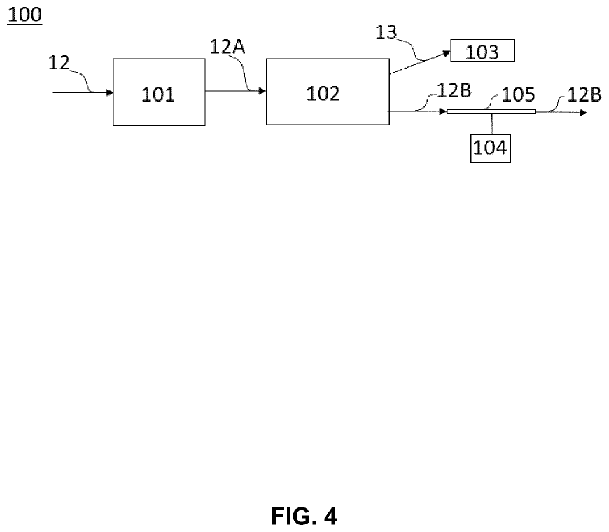

As shown in the non-limiting example of FIG. 4, some embodiments of the present disclosure relate to the processing unit 100 comprising a heatable tank 101, a degassing chamber 102 and a condenser 103. The heatable tank 101 can be configured to receive an input mixture, which may be input mixture 12. In some embodiments of the present disclosure the heatable tank 101 may be heated and, optionally, under vacuum for producing a heated input mixture 12A. The heated input mixture 12A (comprising one or more cannabinoids) may be conducted to the degassing chamber 102 that may also be operated under vacuum. Those skilled in the art will appreciate that input mixture 12 may be introduced to degassing chamber 102, or not. In some embodiments of the present disclosure, the condenser 103 may be used to condense one or more gaseous compounds 13 that are removed by the degassing chamber 102. The one or more gaseous compounds 13 can be condensable or non-condensable gases. Some examples of condensable gases include, but are not limited to: solvent residue (ethanol, butane and the like), volatile organic compounds, such as terpenes and water. In an embodiment, the primary non-condensable gas is CO₂. In an embodiment, the processing unit 100 may also comprise a transfer pump 104 and a heated hose 105 that can be configured to conduct the processed second input mixture 12B to either to the first end 310A of the column 310, the middle of the column 310, the volatizing unit 200, or any combination thereof.

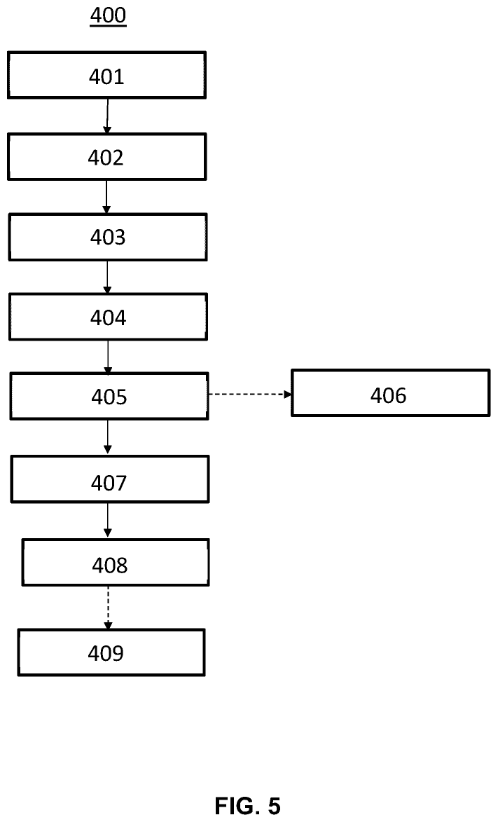

FIG. 5 shows a sequence of steps that form part of an embodiment of a method 400 that relates to separating a cannabinoid from an input mixture according to embodiments of the present disclosure.

The method 400 comprises a step 401 of volatilizing the input mixture 12 to provide a cannabinoid-containing vapor stream 14 and a residue 16.

The method 400 further comprises a step 402 of conducting the cannabinoid-containing vapor stream to a fractional distillation unit 300 and a step 403 separating a first cannabinoid from at least a second cannabinoid within the fractional distillation unit 300. In some embodiments of the present disclosure, the fractional distillation unit 300 may be a fractional distillation unit that operates continuously. The fractional distillation unit 300 may be operated at specific temperatures and pressures while performing a step 403 of separating a first cannabinoid from at least a second cannabinoid within the fractional distillation unit 300.

The method 400 further comprises a step 404 of conducting a portion of the cannabinoid-containing vapor stream comprising the first cannabinoid to a condenser unit and a step 405 of condensing the conducted portion of the cannabinoid-containing vapor stream within the condenser unit to provide a condensed first cannabinoid product stream and a recirculate stream.

The method 400 may optionally comprise a step 406 of repeating steps 401 to 405 using the recirculate stream.

The method 400 further comprises a step 407 of collecting a product from the fractional distillation unit, wherein the product comprises at least the second cannabinoid.

The method 400 may further comprise a step 408 of the conducting the recirculate stream to the fractional distillation unit.

In an embodiment, the method 400 may comprise a step 409 of conducting a non-condensed portion of the preliminary cannabinoid-containing product stream back to mix with the contents of the fractional distillation column 310. The method 400 further comprises 408 of collecting a product stream that contains at least a first cannabinoid from the fractional distillation unit. The steps 406 and 407 of the method 400 may be repeated once or more than once before collection the step 408, or not.

### EXAMPLES

### Example 1—Input Mixture

The input mixture containing cannabinoids may be a cannabis resin that was prepared by first preparing a precursor extract by the steps of dissolution, chilling and filtering to produce the precursor extract, which may be substantially transparent. Optionally, the precursor extract can be substantially or completely depleted of lipids and waxes. The precursor extract can have a cannabinoid concentration of between about 60% and about 90% (wt/wt) or between about 70% and about 80% (wt/wt).

The precursor extract can be heated to between about 130° C. and about 160° C. while being stirred to produce the input mixture.

### Example 2—Separation

The input mixture of Example 1 was introduced into the wiped film evaporator unit (i.e. a volatizing unit). The cannabinoids were evaporated from the input mixture into a cannabinoid-containing vapor stream inside the wiped film evaporator unit and the cannabinoid-containing vapor stream was conducted to the fractional distillation unit.

The cannabinoid-containing vapor stream was conducted through the fractional distillation column where at least one cannabinoid in the vapor stream was separated from the other constituents of the vapor stream. A product was collected from the distillation unit and non-volatile components were discharged from the distillation unit.

### Example 3—Short Path Distillation

In instances where the input mixture comprises volatile organic compounds (VOCs) and/or acidic cannabinoids, the input mixture may not be suitable for direct input into a short path distillation unit as the volatizing unit. This is because the VOCs can off gas and interfere with the vacuum pressure of the short path distillation unit. Additionally, the acidic cannabinoids can decarboxylate at the distillation temperatures contemplated herein, which generates CO₂ that can also interfere with the vacuum pressure of the short path distillation unit. Instead, this type of input mixture can be subjected to a two-staged process that includes a first degassing step and a second short path distillation step and then subjected to the fractional distillation process.

The first step includes degassing by heating these types of input mixtures to about 100° C. in a feed hopper and passing the heated input mixture through a 0.25 cubic foot horizontal wipe-film evaporator that is operating at a temperature of about 150° C. and at feed speed of about 150 mL per minute. The vacuum is maintained at about 0.5 atm absolute with appropriately sized condenser set to about 4° C. to collect any volatile compounds that are evaporated. This process effectively strips the input mixture of some or substantially all volatile compounds or acidic cannabinoids that may interfere later with shortpath distillation. Alternatively, the input mixture may be heated on a hotplate to about 150° C. with stirring until no more gas is produced.

After this degassing, the input mixture is maintained at about 100° C. and pumped into the shortpath distillation system where it is heated to between about 120° C. and about 250° C.—depending on the operating pressure—causing the cannabinoids to evaporate. The evaporated cannabinoids can be directed to the fractional distillation unit or they may be re condensed on an internal condenser set at about 50° C. and about 60° C. and any remaining low boiling point compounds are collected using a cold trap that is at about −40° C. ahead of the vacuum pump.

### Example 4—Short Path Distillation for Enrichment of CBD at Various Temperatures

An input mixture was prepared using a plant input that had on average 14 wt/wt % CBD and <1 wt/wt THC. The input mixture was subjected to a two-step shortpath distillation process (using the VKL70-5 Shortpath distillation system), as described herein above and then subjected to a distillation process at temperatures ranging between about 129° C. and 169° C. The cannabinoid concentration for the residue product (RP) and the distillation product (DP) are provided below in Table 1.

TABLE 1
Cannabinoid concentration of short path distilled and further distilled input mixture.

| | Cannabinoid Concentration | | | | | | |
|---|---|---|---|---|---|---|---|
| | | | | | | | Residue |
| Temperature | | | RP Total | | | DP Total | Ratio: |
| (° C.) | RP CBD | RP THC | Cannabinoids | DP CBD | DP THC | Cannabinoids | CBD/THC |
| 169 | 3.39 | 0.38 | 3.77 | 67.72 | 2.67 | 75.15 | 8.92 |
| 164 | 2.53 | 2.49 | 82.15 | 82.15 | 2.95 | 89.87 | 14.32 |
| 159 | 3.64 | 3.64 | 81.33 | 81.33 | 2.58 | 88.56 | 12.74 |
| 154 | 4.52 | 4.52 | 82.27 | 82.27 | 2.42 | 88.53 | 13.76 |
| 149 | 3.06 | 3.06 | 83.28 | 83.28 | 2.28 | 89.48 | 27.80 |
| 144 | 3.21 | 3.21 | 88.33 | 88.33 | 2.03 | 94.42 | 28.45 |
| 139 | 2.59 | 2.59 | 82.81 | 82.81 | 1.41 | 87.5 | 33.97 |
| 134 | 1.75 | 1.75 | 81.87 | 81.87 | 0.95 | 85.8 | 49.33 |
| 129 | 1.33 | 1.33 | 78.62 | 78.62 | 0.92 | 82.58 | 68.60 |

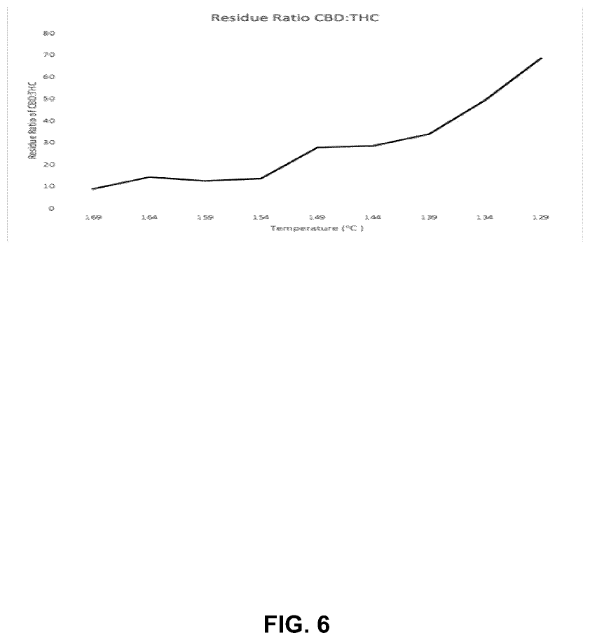

FIG. 6 shows a line graph that represents the RP ratio of CBD:THC plotted over the various distillation temperatures. Without being bound by any particular theory, THC can be distilled out to produce a higher concentration of CBD in a RP as shown by an increasing the ratio of CBD:THC to about 8× when temperatures are at about 129° C. as compared to higher temperatures.

### Example 5—Short Path Distillation for Enrichment of CBD at Various Pressures

In this example, a second pass distillate was passed through a short path distillation unit (as described above in Example 5) and then subjected to the fractional distillation process at various pressures (2.6-3.0 mbar) and various temperatures (about 169° C. to about 234° C.) in order to assess the evaporation points and the purity of the resulting distillate and residue. Table 2 summarizes the residue/distillate accumulation over the various temperatures utilized.

TABLE 2
Residue and Distillate Accumulation Data

| | Residue/Distillate Accumulation | | | |
|---|---|---|---|---|
| Temperature | Input | Residue | Distillate | Distillate/ |
| (° C.) | Weight (g) | Weight (g) | Weight (g) | Residue Split |
| 169 | 269.4 | 230.0 | 0 | 0 |
| 179 | 230.0 | 218.1 | 5.0 | 0.02 |
| 189 | 222.5 | 199.3 | 22.8 | 0.11 |
| 194 | 218.8 | 189.3 | 27.9 | 0.15 |
| 199 | 216.0 | 176.1 | 41.8 | 0.26 |
| 204 | 219.6 | 166.6 | 47.3 | 0.28 |
| 209 | 213.9 | 149.3 | 67.1 | 0.44 |
| 214 | 216.3 | 106.6 | 107.8 | 1.01 |
| 219 | 211.8 | 97.5 | 116.5 | 1.19 |
| 224 | 210.6 | 47.1 | 162.9 | 3.45 |
| 229* | 206.2 | 50.8 | 153.1 | 3.01 |
| 229 | 202.8 | 22.4 | 143.7 | 6.42 |
| 234* | 162.4 | 45.6 | 81.5 | 1.79 |
| 234 | 123.4 | 7.7 | 105.0 | 13.63 |

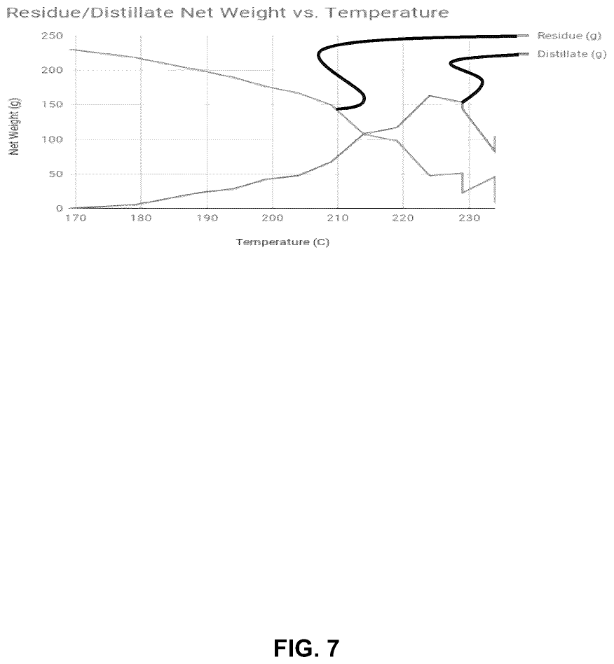

FIG. 7 shows a line graph that represents the net weight versus the temperature of the residue and the distillate, respectively.

Without being bound by any particular theory, the ratio of distillate to residue increased dramatically at about 214° C. This suggests that this may represent an optimal temperature point for evaporation of cannabinoids at 2.6-3 mbar. Higher temperatures were seen to cause burning of material on the inside of the distillation chamber.

### Example 6—Constant Temperature and Constant Feed Rate

An input mixture was subjected to similar experimental conditions as described above in Example 6, but the distillation process was conducted at a constant temperature of 134° C. at a feed rate of 15 Hz. Table 3 summarizes the cannabinoid concentration of the RP and the DP.

TABLE 3
Cannabinoid Concentration of Residue Product and Distillate Product

| | | Cannabinoid Concentration | | | | | | | |
|---|---|---|---|---|---|---|---|---|---|
| Trial | Temperature | RP | RR | RP Total | DP | DP | DP Total | Ratio: | |
| # | (° C.) | CBD | THC | Cannabinoids | CBD | THC | Cannabinoids | CBD/THC | Enrichment |
| 1 | 134 | 28.43 | 23.70 | 57.61 | 50.01 | 20.97 | 75.94 | 2.38 | 1.41 |
| 2 | 134 | 44.84 | 31.71 | 82 61 | 57.63 | 18.19 | 80.20 | 3.16 | 1.33 |
| 3 | 134 | 63.14 | 19.79 | 87.33 | 65.87 | 16.68 | 86.56 | 3.95 | 1.25 |
| 4 | 134 | 62.56 | 24.35 | 91.76 | 71.44 | 14.55 | 89.63 | 4.91 | 1.24 |

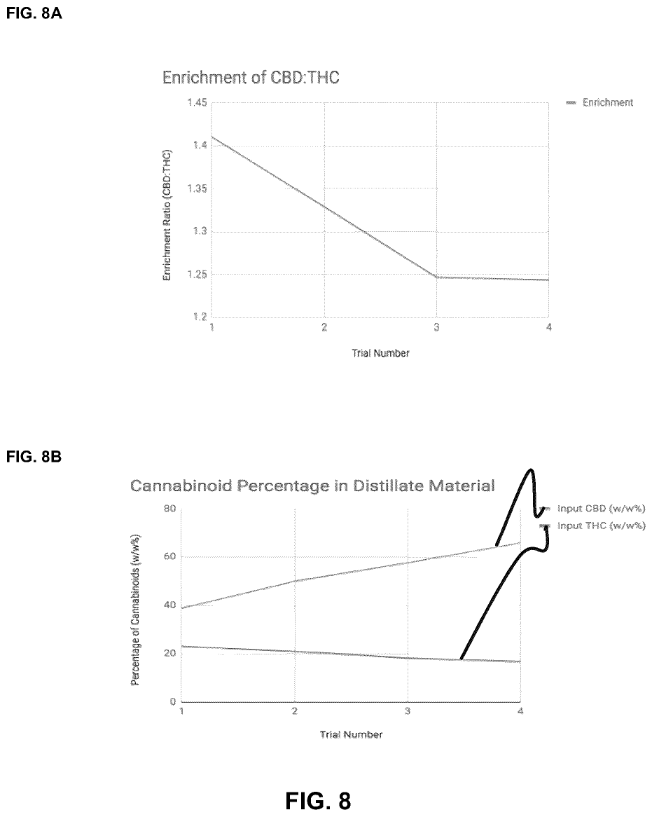

FIG. 8A shows a line graph that represents the enrichment of CBD:THC over the four trials. FIG. 8B shows a line graph that represents the percentage of CBD (upper line) and THC (lower line) in the distillate product.

Without being bound by any particular theory, these results indicate that CBD can be enriched in the distillate product at constant temperature and pressure.

### Example 7—Variable Feed Rates

An input mixture was subjected to similar experimental conditions as described above in Example 6, but the distillation process was conducted at a constant temperature of 209° C. at a feed rate of between about 2.5 Hz and about 10 Hz and at pressure of between about 8 mmHg and 10 mmHg. Table 4 summarizes the residue/distillate accumulation over the various temperatures utilized.

TABLE 4
Residue and Distillate Accumulation Data

| | Residue/Distillate Accumulation | | | | |
|---|---|---|---|---|---|
| Pressure | Input Net | Residue | Distillate | Output Net | Distillate/ |
| (mmHg) | Weight (g) | Weight (g) | Weight (g) | Weight (g) | Residue Split |
| 2 | 213.9 | 149.3 | 67.1 | 216.4 | 0.45 |
| 8 | 610.3 | 411.4 | 194.2 | 605.6 | 0.47 |
| 10 | 596.5 | 379.6 | 187.1 | 566.7 | 0.49 |

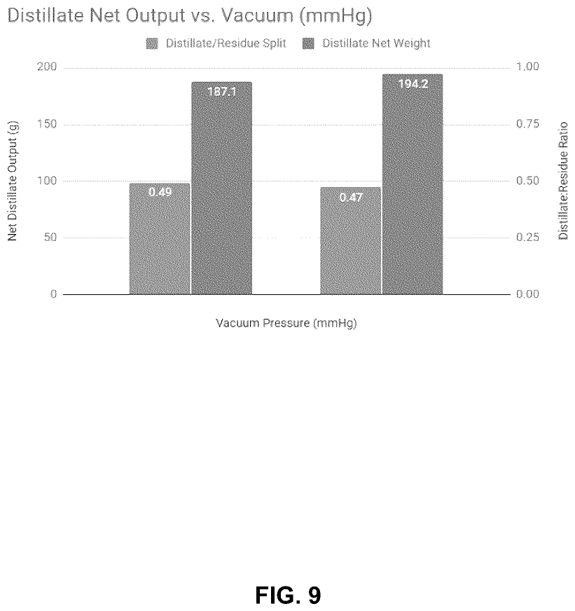

FIG. 9 shows a histogram that represents the distillate net output compared with the pressure within the distillation unit.

Without being bound by any particular theory, the ratio of distillate to residue at 210° C. with a feed rate of 10 Hz remained fairly constant even when the feed rate was decreased to 8 mmHg. Also, when compared to 209° C. at 2 mmg with a feed rate of 25 Hz, the spit ratio remained substantially constant.

Table 5 shows the accumulation of residue product and distillate produce at various feed rates (2.5 Hz, 5 Hz and 10 Hz).

TABLE 5
Residue and Distillate Accumulation Data

| | Residue/Distillate Accumulation | | | | |
|---|---|---|---|---|---|
| Feed Rate | Input Net | Residue | Distillate | Output Net | Distillate/ |
| (Hz) | Weight (g) | Weight (g) | Weight (g) | Weight (g) | Residue Split |
| 10 | 676.2 | 589.3 | 59.0 | 648.3 | 0.10 |
| 5.0 | 610.3 | 411.4 | 194.2 | 605.6 | 0.47 |
| 2.5 | 314.5 | 28.9 | 276.1 | 305.0 | 9.55 |

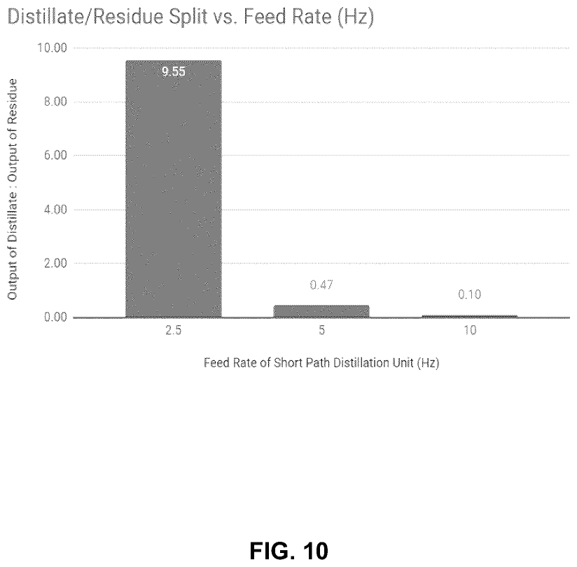

FIG. 10 shows a histogram that represents the distillate/residue split as compared to the feed rate. When using a substantially constant temperature of about 210° C. and a vacuum pressure of about 10 mmHg (13.2-13.4 mbar) for the Short Path Distillation of a Second pass resin, it was found that the most efficient feed speed to promote the greatest evaporation and distillation is about 2.5 Hz. At 2.5 Hz, the material distills over at a distillate to residue ratio of 9.55. Without being bound by any particular theory, this data demonstrates that there may be a significant time to allow for evaporation and distillation of the material. Further, the other parameters (210° C. and 10 mmHg) still promote evaporation within the evaporation chamber without degrading the cannabinoids.

### Example 8—Omega 3 Oil

Instead of the input mixture, a mixture of omega 3 oils was subjected to a similar set of experimental conditions as Example 6. There were variable temperatures, a substantially constant pressure and a feed rate of about 89 mL/hr. Table 6 shows the accumulation of residue product and distillate product at various temperatures.

TABLE 6
Residue and Distillate Accumulation Data

| | Resid ue/Distillate Accumulation | | | | | Percent |
|---|---|---|---|---|---|---|
| Temperature | Input Net | Residue | Distillate | Output Net | Distillate/ | Distillate |
| (° C.) | Weight (g) | Weight (g) | Weight (g) | Weight (g) | Residue Split | (%) |
| 160 | 136 | 83 | 22.2 | 105.2 | 0.27 | 21.1 |
| 165 | 74.7 | 38.6 | 32.4 | 71.0 | 0.84 | 45.6 |
| 170 | 76.1 | 28.6 | 46.3 | 74.9 | 1.62 | 61.8 |
| 175 | 44.4 | 3.3 | 38.2 | 41.6 | 11.61 | 93.3 |

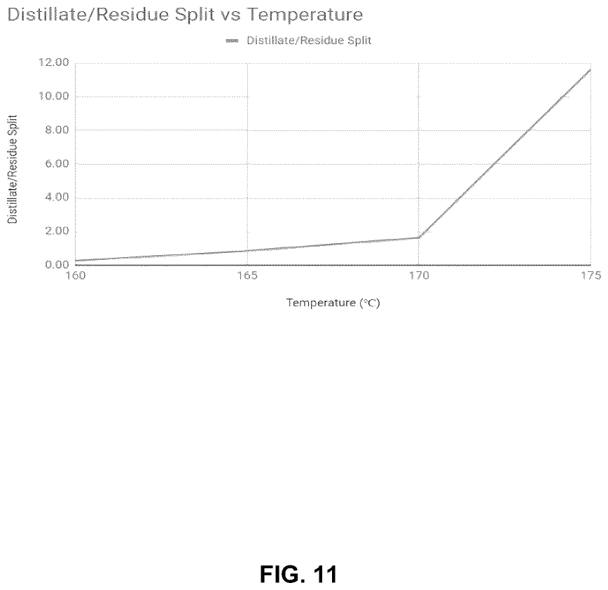

FIG. 11 shows a line graph that represents the distillate/residue split as compared to the various temperatures tested. It was found that at a temperature of about 175° C., over 93% of the Omega-3 oil INPUT was distilled. A temperature of about 175° C., at a feed speed of about 2.5 Hz and pressure of 10 mmHg produced a distillate to residue ratio of 11.61.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. An apparatus for isolating one or more cannabinoids from an input mixture, the apparatus comprising:
(a) a volatizing unit that is configured to receive and volatilize the input mixture into a cannabinoid-containing vapor stream and a residue;
(b) a fractional distillation unit that is in fluid communication with the volatizing unit, the fractional distillation unit comprising a column that defines a plenum for receiving the cannabinoid-containing vapor stream, wherein the plenum is configured to separate a first cannabinoid within the cannabinoid-containing vapor stream from at least a second cannabinoid; and
(c) a condenser unit that is configured to receive a portion of the cannabinoid-containing vapor stream comprising the first cannabinoid from the plenum and to form a condensed first cannabinoid output stream and a recirculate stream.

2. The apparatus of claim 1, further comprising a distributor that is configured to receive the recirculate stream and to conduct the recirculate stream to the plenum.

3. The apparatus of claim 1, wherein the volatizing unit is a wiped film evaporator, a short path distillation unit, a rising-falling film evaporator, a pot still, a jacketed or heated vessel, a centrifugal evaporator, a centrifugal short path distillation, or any combination thereof.

4. (canceled)

5. The apparatus of claim 1, wherein the fractional distillation unit is a continuous fractional distillation unit.

6. The apparatus of claim 1, wherein the plenum is configured to separate the first cannabinoid from the second cannabinoid and at least one or more additional cannabinoids.

7. (canceled)

8. (canceled)

9. (canceled)

10. The apparatus of claim 6, wherein the plenum is configured to separate the second cannabinoid from at least one of the one or more additional cannabinoids.

11. (canceled)

12. The apparatus of claim 1, wherein the input mixture is a cannabis resin.

13. The apparatus of claim 1, wherein the first cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

14. The apparatus of claim 13, wherein the first cannabinoid or the second cannabinoid is THC.

15. The apparatus of claim 13, wherein the first cannabinoid or the second cannabinoid is CBD.

16. A method for isolating one or more cannabinoids from an input mixture, the method comprising steps of:
(a) volatilizing the input mixture to provide a cannabinoid-containing vapor stream and a residue;
(b) conducting the cannabinoid-containing vapor stream to a fractional distillation unit;
(c) separating a first cannabinoid from at least a second cannabinoid within the fractional distillation unit;
(d) conducting a portion of the cannabinoid-containing vapor stream comprising the first cannabinoid to a condenser unit;
(e) condensing the conducted portion of the cannabinoid-containing vapor stream within the condenser unit to provide a condensed first cannabinoid product stream and a recirculate stream;
(f) optionally repeating steps (a) to (e) using the recirculate stream; and
(g) collecting a product from the fractional distillation unit, wherein the product comprises at least the second cannabinoid.

17. The method according to claim 16, wherein the volatilizing is by wiped film evaporation.

18. The method according to claim 16, further comprising a step of conducting the recirculate stream to the fractional distillation unit.

19. The method according to claim 18, wherein the step of conducting the recirculate stream further comprises conducting the recirculate stream to and through a distributor before conducting the recirculate stream to the fractional distillation unit.

20. The method according to claim 16, wherein the step of separating the first cannabinoid further comprises a step of separating the first cannabinoid from the second cannabinoid and at least one or more additional cannabinoids.

21. (canceled)

22. The method according to claim 16, wherein the input mixture is a cannabis concentrate.

23. The method according to claim 16, wherein the input mixture is a cannabis resin.

24. The method according to claim 16, wherein the first cannabinoid and the at least second cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

25. The method according to claim 24, wherein the first cannabinoid or the at least second cannabinoid is THC.

26. The method according to claim 24, wherein the first cannabinoid or the at least second cannabinoid is CBD.
